(19) United States
(12) Patent Application Publication
(10) Pub. No.: US 2022/0241007 A1
(43) Pub. Date: Aug. 4, 2022

(54) ELECTROSURGICAL TISSUE SEALING DEVICE WITH NON-STICK COATING

(71) Applicants: Kester Julian Batchelor, Mound, MN (US); Teo Heng Jimmy Yang, Heath (GB); Riyad Moe, Madison, WI (US)

(72) Inventors: Kester Julian Batchelor, Mound, MN (US); Teo Heng Jimmy Yang, Heath (GB); Riyad Moe, Madison, WI (US)

(21) Appl. No.: 17/585,192

(22) Filed: Jan. 26, 2022

Related U.S. Application Data
(60) Provisional application No. 63/143,345, filed on Jan. 29, 2021.

(51) Int. Cl. A61B 18/14

(52) U.S. Cl. CPC A61B 18/1445; A61B 2018/0013

(57) ABSTRACT

An electrosurgical instrument includes a jaw member having an electrically conductive tissue sealing plate configured to operably couple to a source of electrosurgical energy for treating tissue. A non-stick coating formed front a liquidphobic structure is deposited to at least a portion of the electrically conductive sealing plate to reduce tissue adherence during application of electrical energy to tissue.

### CROSS REFERENCE TO RELATED APPLICATION

This application claims priority to U.S. Provisional Patent Application No. 63/143,345, titled “ELECTROSURGICAL TISSUE SEALING DEVICE WITH NON-STICK COATING.”, filed on Jan. 29, 2021, the contents of which are hereby incorporated by reference.

### TECHNICAL FIELD

This document pertains generally, but not by way of limitation, to electrosurgical devices that can be used for various surgical procedures.

### OVERVIEW

Electrosurgical forceps utilize mechanical clamping action along with electrical energy to effect hemostasis on the clamped tissue. The forceps (open, laparoscopic or endoscopic) include electrosurgical sealing plates which apply the electrosurgical energy to the clamped tissue. By controlling the intensity, frequency, and duration of the electrosurgical energy applied through the sealing plates to the tissue, the surgeon can coagulate, cauterize, and/or seal tissue.

In the past, significant efforts have been directed to improvements in electrosurgical instruments and the like, with a view towards providing improved transmission of electrical energy to patient tissue in both an effective manner and to reduce the sticking of soft tissue to the instrument's surface during application. In general, such efforts have envisioned non-stick surface coatings, such as polymeric materials, for increasing the lubricity of the tool surface. However, these materials may interfere with the efficacy and efficiency of hemostasis and have a tendency to release from the instrument's substrate due to formation of microporosity, delamination, and/or abrasive wear, thus exposing underlying portions of the instrument to direct tissue contact and related sticking issues. In turn, these holes or voids in the coating lead to nonuniform variations in the capacitive transmission of the electrical energy to the tissue of the patient and may create localized excess heating, resulting in tissue damage, undesired irregular sticking of tissue to the electrodes.

The present inventors have recognized, among other things, that problems to be solved in using electrosurgical devices is to provide a non-stick coating to minimize undesired irregular sticking or damage to tissue while providing benefits. For example, the present inventors have recognized that providing a non-stick coating of a liquidphobic structure, e.g., a hydrophobic or superhydrophobic coating can provide tissue adhesion resistance.

In one example of the present disclosure, an electrosurgical instrument is provided and includes at least one jaw member having an electrically conductive tissue sealing plate configured to operably couple to a source of electrosurgical energy for treating tissue and a non-stick coating formed from a liquidphobic structure. In one example, the liquidphobic structure includes fluorosilane containing compounds.

In one example of the present disclosure, an electrosurgical instrument is provided and includes at least one jaw member having an electrically conductive tissue sealing plate configured to operably couple to a source of electrosurgical energy for treating tissue and a non-stick coating formed from a liquidphobic structure. In one example, the liquidphobic structure includes fluorosilane containing compounds and has a thickness of from about 10 nanometers to about 300 nanometers disposed on at least a portion of the tissue sealing plate. A thickness of about 10 nm can provide a minimum level of non-stick performance and durability, and depending on the device and the number of intended uses, 20 nm may be more preferred. A thickness of about 300 nanometers can provide improved non-stick performance and durability over thinner coatings of the about 10-20 nm range. Above about 300 nanometers, additional performance and durability enhancements may not be realized, while additional cost is incurred. Further, depending on the particular device characteristics, coating thicknesses above 300 nm may undesirably affect electrical transmission from the tissue sealing plate to the tissue. Thus, in a possibly preferred example, the liquidphobic structure can include fluorosilane containing compounds less than the maximum of about 300 nanometers, such as having a thickness in a range from about 10 nanometers to about 200 nanometers, or more preferably in an range from about 20 nanometers to about 200 nanometers to provide the performance, durability and value. In one example, the non-stick coating is formed from perfluoropolyether (PFPE).

In one example, the non-stick coating has a substantially uniform thickness. In another example, the non-stick coating has a non-uniform thickness. In another example, the non-stick coating is discontinuous. In another example, the non-stick coating is continuous. In another example, the electrosurgical instrument also includes an insulative layer disposed on at least a portion of the tissue sealing plate. In another example, the non-stick coating is disposed on at least a portion of each of the pair of opposing jaw members. In another example, the tissue sealing plate is formed of stainless steel.

According to another example of the present disclosure, an electrosurgical instrument is provided and includes a pair of opposing jaw members. Each of the opposing jaw members includes an electrically conductive tissue sealing plate configured to operably couple to a source of electrosurgical energy for treating tissue, a support base configured to support the tissue sealing plate, and an insulative housing configured to secure the tissue sealing plate to the support base. A non-stick coating formed from a liquidphobic structure is disposed on at least a portion of at least one of the opposing jaw members. In one example, the non-stick coating is disposed on at least a portion of each of the tissue sealing plates, the support base, and the insulative housing. In one example, the non-stick coating thickness has a substantially uniform thickness on the tissues sealing plates, the support base, and the insulative housing. In another example, the coating has a non-uniform thickness. In another example, the non-stick coating is discontinuous. In another example, the non-stick coating is continuous.

According to another example of the present disclosure, an electrosurgical instrument is provided and includes a pair of opposing jaw members. Each of the opposing jaw members includes an electrically conductive tissue sealing plate configured to operably couple to a source of electrosurgical energy for treating tissue, a support base configured to support the tissue sealing plate, and an insulative housing configured to secure the tissue sealing plate to the support base. A non-stick coating formed from a liquid phobic structure is disposed on at least a portion of at least one of the opposing jaw members. In one example, the non-stick coating is disposed on at least a portion of each of the tissue sealing plates, the support base, and the insulative housing. In one example, the non-stick coating thickness has a substantially uniform thickness on the tissues sealing plates, the support base, and the insulative housing. In another example, the coating has a non-uniform thickness. In another example, the non-stick coating is discontinuous. In another example, the non-stick coating is continuous.

According to another example of the present disclosure, a method of inhibiting tissue from sticking to an electrically conductive component of an electrosurgical tissue sealing device during application of energy to tissue is provided. The method includes applying a non-stick coating on at least a portion of an electrically conductive component of an electrosurgical tissue sealing device. The method also includes controlling a thickness of the non-stick coating applied to inhibit tissue from sticking to the electrically conductive component during application of energy to the tissue. The thickness of the non-stick coating also allows a sensing of at least one tissue parameter generated via application of energy to the tissue.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a side view of an electrosurgical device having jaws in an open configuration, according to an example of the present application.

FIG. 2A is a side view of a portion of an electrosurgical device including the jaws, in accordance with at least one example.

FIG. 2B is a perspective view of the portion of the electrosurgical device in FIG. 2A.

FIG. 3 illustrates an expanded view of a jaw member, in accordance with at least one example.

FIG. 4 illustrates a jaw member including a non-stick coating, in accordance with at least one example.

FIG. 5 illustrates a jaw member including a non-stick coating, in accordance with at least one example.

FIG. 6 illustrates a jaw member including a non-stick coating, in accordance with at least one example.

FIG. 7 illustrates a jaw member including a non-stick coating, in accordance with at least one example.

FIG. 8 illustrates a jaw member including a non-stick coating, in accordance with at least one example.

FIG. 9 illustrates a jaw member including a non-stick coating, in accordance with at least one example.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

As described in more detail below with reference to the accompanying figures, the present disclosure is directed to electrosurgical devices having a non-stick coating formed from a liquidphopic structure disposed on one or more components (e.g., tissue sealing plates, jaw members, electrical leads, insulators etc.). The thickness of the non-stick coating is controlled, allowing for desired electrical performance, while providing tissue sticking reduction during tissue sealing. As discussed herein, the present inventors have determined that fluorosilanes can provide non-stick properties.

As used herein, “liquidphobic” or “super-liquidphobic” structures describe, in a general sense, any material that displays anti-liquid properties, e.g., a material that is one or more of hydrophobic (repels water), lipophobic (repels oils and lipids), amphiphobic (a material which is both hydrophobic and lipophobic), hemophobic (repels blood or blood components) or the like. Such materials repel liquids, e.g., by causing the liquid to bead-up on the material's surface and not spread out or wet the material's surface. Thus, as used herein, a substrate that is described as comprising a liquidphobic structure includes substrates that comprise a liquidphobic, super-liquidphobic, hydrophobic, super-hydrophobic, amphiphobic and/or super-amphiphobic substrate.

When a drop of a liquid (e.g., water based, lipid based, etc.) rests upon a surface, it will spread out over the surface to a degree based upon such factors as the surface tensions of the liquid and the substrate, the smoothness or roughness of the surface, etc. For example, the liquidphobicity of a substrate can be increased by various coatings that lower the surface energy of the substrate. The quantification of liquidphobicity can be expressed as the degree of contact surface angle (or contact angle) of the drop of the liquid on the surface.

For example, for a surface having a high surface energy (i.e., higher than the surface tension of the liquid drop), a drop of liquid will spread out “wetting” the surface of the substrate. Such surface displays liquidphilicity, as opposed to liquidphobicity. When the surface energy of a substrate is decreased, liquidphobicity is increased (and vice versa). Liquidphobic, including hydrophobic, lipidphobic and/or amphiphobic refer to properties of a substrate which cause a liquid drop on their surface to have a contact angle of 90 degrees (°) or greater. “Super-hydrophobicity,” “super-amphiphobicity,” and “super-liquidphobicity” all refer to properties of substances which cause a liquid drop on their surface to have a contact angle of 150° or greater.

The liquidphobic structures, when applied to electrosurgical devices, can reduce the sticking of tissue during the application of electrosurgical energy for treating tissue. For example, the superhydrophobicity texture consists of an array of micro pillars that supports the water droplets (can be saline or other liquid) and not adhere to the surface. In contrast, a substrate without the micro-pillar allows the water droplets to spread across the surface.

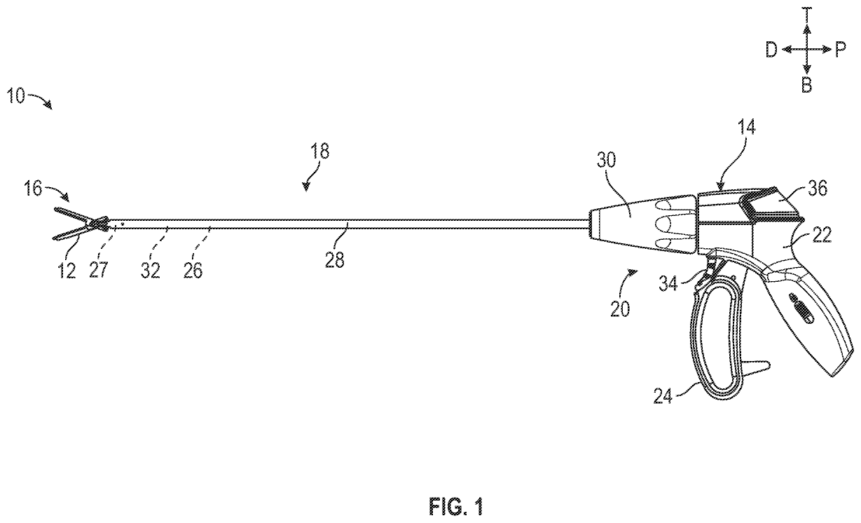

FIG. 1 illustrates a side view of a forceps 10 with jaws 12 in an open position. Directional descriptors such as proximal and distal are used within their ordinary meaning in the art. The proximal direction P and distal direction D, as well as top T and bottom B, are indicated on the axes provided in FIG. 1. The forceps can include a handpiece 14, one or more actuators 20, an outer shaft 28 (or outer tube), an inner shaft 26 (or inner tube), a drive bar 27, and an end effector 16.

The forceps 10 can include the handpiece 14 at a proximal end and the end effector 16 at a distal end. An intermediate portion 18 can extend between the handpiece 14 and the end effector 16 to operably couple the handpiece 14 to the end effector 16. Various movements of the end effector 16 can be controlled by one or more actuation systems 20 of the handpiece 14. In the illustrative example, the end effector 16 can include the jaws 12 that are capable of moving between an open position and a closed position. The end effector 16 can be rotated along a longitudinal axis of the forceps 10. The end effector 16 can include a cutting blade and an electrically conductive tissue sealing plate, e.g., an electrode, for applying electrosurgical energy.

The forceps 10 can include the jaws 12, a housing 22, a lever 24, the inner shaft 26, the drive bar 27, the outer shaft 28, a rotational actuator 30, a blade 32, a trigger 34 and/or an activation button 36. In this example, the end effector 16, or a portion of the end effector 16 can be one or more of: opened, closed, rotated, extended, retracted, and electrosurgically energized.

To operate the end effector 16, the user can displace the lever 24 proximally to drive the jaws 12 from the open position (FIG. 1) to a closed position. In the example of forceps 10, moving the jaws 12 from the open position to the closed position allows a user to clamp down on and compress a tissue. The handpiece 14 can also allow a user to rotate the end effector 16. For example, rotating rotational actuator 30 causes the end effector 16 to rotate by rotating both the inner shaft 26, drive bar 27, and the outer shaft 28 together.

In some examples, with the tissue compressed, a user can depress the activation button 36 to cause an electrosurgical energy to be delivered to the end effector 16, such as to an electrode. Application of electrosurgical energy can be used to treat the tissue such as seal or otherwise affect the tissue being clamped. In some examples, the electrosurgical energy can cause tissue to be sealed, ablated, and/or coagulated. Electrosurgical energy can be applied to any suitable electrode.

In some examples, the forceps 10 can be used to cut the treated tissue via a blade assembly 32 (also referred to as blade 32). For example, the handpiece 14 can enable a user to extend and retract the blade 32. The blade 32 can be extended by displacing the trigger 34 proximally. The blade 32 can be retracted by allowing the trigger 34 to return distally to a default position. The default position of the trigger 34 is shown in FIG. 1. In some examples, the handpiece 14 can include features that inhibit the blade 32 from being extended until the jaws 12 are at least partially closed, or fully closed.

The forceps 10 can be used to perform a treatment on a patient, such as a surgical procedure. In an example, a distal portion of the forceps 10, including the jaws 12, can be inserted into a body of a patient, such as through an incision or another anatomical feature of the patient's body. While a proximal portion of the forceps 10, including the housing 22 remains outside the incision or another anatomical feature of the body. Actuation of the lever 24 causes the jaws 12 to clamp onto a tissue. The rotational actuator 30 can be rotated via a user input to rotate the jaws 12 for maneuvering the jaws 12 at any time during the procedure. Activation button 36 can be actuated to provide electrical energy to jaws 12 to cauterize, desiccate, or seal the tissue within the closed jaws 12. Trigger 34 can be moved to translate the blade 32 distally in order to cut the tissue within the jaws 12.

Examples of forceps are shown and described in U.S. Patent Application Publication No, 2020/0305960, the entire contents of which are incorporated herein by reference.

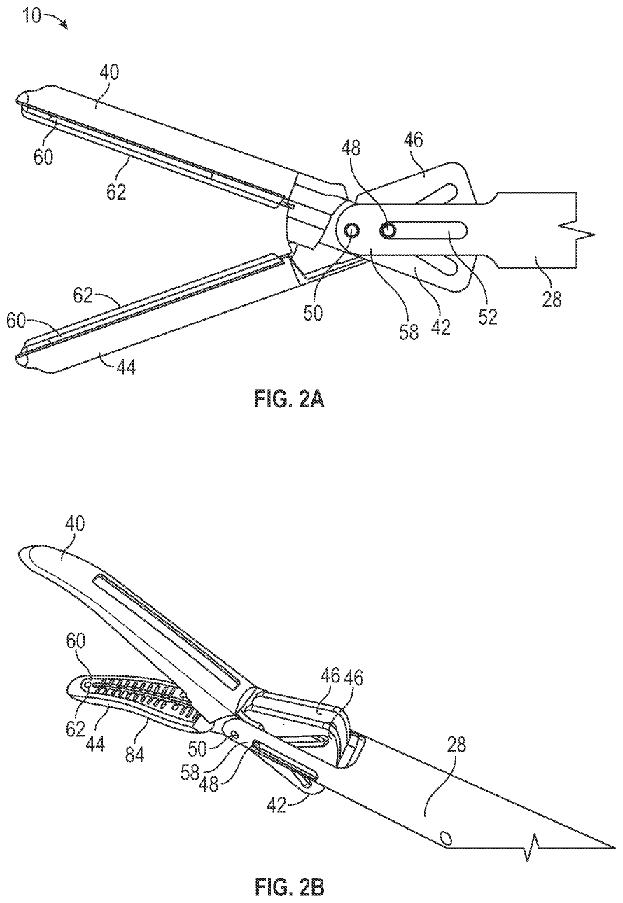

FIG. 2A illustrates a side view of a portion of forceps 10, in accordance with at least one example of this disclosure. FIG. 2B illustrates a perspective view of a portion of the forceps 10. FIGS. 2A and 2B are discussed below concurrently. The forceps 10 can include a top jaw 40 (including flanges 42), a bottom jaw 44 (including flanges 46), a drive pin 48, a pivot pin 50, an inner shaft 52, and an outer shaft 28. The outer shaft 28 can include outer arms 58.

The forceps 10 can be used with various surgical procedures. The end effector 16 includes pair of opposing jaw members 40, 44 that rotate about a pivot pin 50 and that are movable relative to one another to grasp tissue. As seen in FIGS. 2A and 2B, the jaw members 40, 44 include an electrically conductive sealing plate

In one example, a sensor can be disposed on or proximate to at least one of the jaw members 40, 44 of the forceps 10 for sensing tissue parameters (e.g., temperature, impedance, etc.) generated by the application of electrosurgical energy to tissue via the jaw members 40, 44. The sensor may include a temperature sensor, tissue hydration sensor, impedance sensor, optical clarity sensor, or the like. A cable, coupling the forceps 10 to an electrosurgical generator, can transmit sensed tissue parameters as data to the electrosurgical generator having suitable data processing components (e.g., microcontroller, memory, sensor circuitry, etc.) for controlling delivery of electrosurgical energy to the forceps 10 based on data received from the sensor.

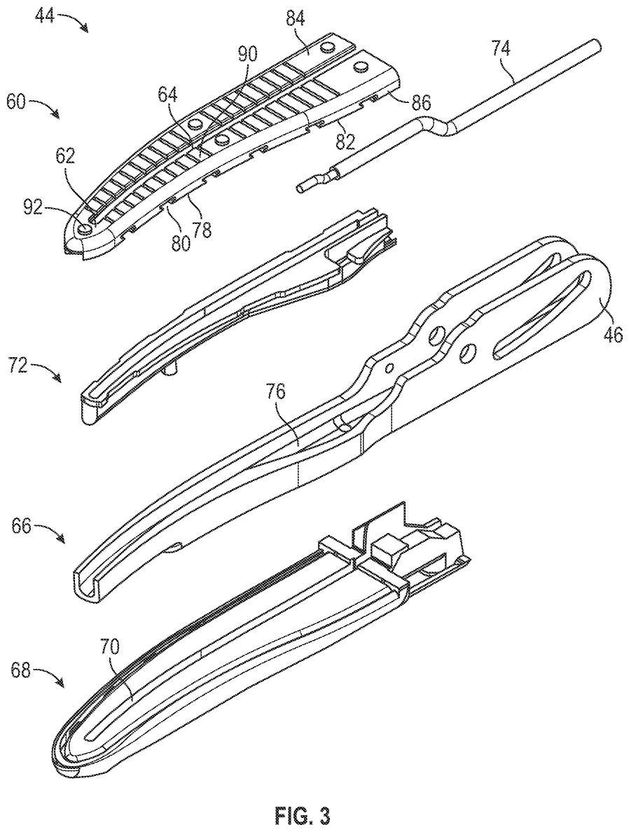

FIG. 3. illustrates an exploded view of the jaw member 44, as shown in FIGS. 2A and 2B. Jaw member 44 can be identical to jaw member 40. The jaw member 44 can include the electrically conductive sealing plate 60, e.g., an electrode, including a blade slot 64, a frame 66 (including flanges 46), an overmold 68 including a blade slot 70, and a support 72. A wire 74 can electrically couple the electrically conductive sealing plate 60 to an energy source.

The overmold 68 can include the blade slot 70, which can be aligned with the blade slot 64 of the electrically conductive sealing plate 60 when the overmold 68 is secured to the electrically conductive sealing plate 60 (such as when the overmold 68 is overmolded to the frame 66 and the electrically conductive sealing plate 60. In an example, the frame 66 can include a slot 76 that can receive the support 72 therein. The support 72 can help to support the electrically conductive sealing plate 60 on the frame 66.

The electrically conductive sealing plate 60 includes an underside surface 82 that can include an electrically insulative layer 86 bonded thereto or otherwise disposed thereon. The electrically insulative layer 86 can electrically insulate the electrically conductive sealing plate 60, from the support 72 and the frame 66. In one example, the electrically insulative layer 86 is formed from polyimide. However, in other examples, any suitable electrically insulative material may be utilized, such as polycarbonate, polyethylene, etc.

Additionally, the jaw member 44 include an external surface 84 that includes a non-stick coating 62 disposed thereon. The non-stick coating 62 may be disposed on selective portions of either of the jaw members 40, 44, or may be disposed on the entire external surface 84. In some examples, the non-stick coating 62 is disposed on a tissue-engaging surface 90 of the electrically conductive sealing plate 60. The non-stick coating 62 is configured to reduce the sticking of tissue to the electrical conducting sealing plates, the jaw members, the electrical leads, and/or the surrounding insulating material.

The support 72 is configured to support the electrically conductive sealing plate 60 thereon. The electrically conductive sealing plate 60 may be affixed atop the support 72 that can be coupled to or integral with the frame 66. The electrically conductive sealing plate 60 can be coupled to the support 72 and/or frame 66, by any suitable method including but not limited to snap-fitting, overmolding, stamping, ultrasonic welding, laser welding, etc. The support 72, frame 66, and the electrically conductive sealing plate 60 is at least partially encapsulated by overmold 68, by way of an overmolding process to secure the electrically conductive sealing plates 60 to the support 72 and the frame 66.

The electrically conductive sealing plate 60 can include teeth 78 that can define recesses 80. The recesses 80 can be located on a side edge of the electrically conductive sealing plate 60. The recesses 80 can be configured to let material of the overmold 68 infiltrate (or fill in) the recesses (or spaces or gaps) 80 so that the electrically conductive sealing plate 60 is secured to the overmold 68. In an example, the electrically conductive sealing plate 60 is an electrode (or can include an electrode) which can be electrically connected to the wire (or conduit) 74.

The electrically conductive sealing plate 60 is coupled to wire 74 (e.g., electrical lead/conduit), via any suitable method (e.g., ultrasonic welding, crimping, soldering, etc.). The wire 74 serves to deliver electrosurgical energy (e.g., from an electrosurgical energy generator) to the electrically conductive sealing plate 60.

Jaw member 44 may also include a series of stop members 92 disposed on the tissue-engaging surface of the electrically conductive sealing plate 60 to facilitate gripping and manipulation of tissue and to define a gap between the jaw members 40, 44 during sealing and cutting of tissue. The series of stop members 92 may be disposed (e.g., formed, deposited, sprayed, affixed, coupled, etc.) onto the electrically conductive sealing plate 60 during manufacturing. Some or all of the stop members 92 may be coated with the non-stick coating 62 or, alternatively, may be disposed on top of the non-stick coating 62.

As discussed herein, the non-stick coating is applied to portions of the electrosurgical device to provide tissue adherence resistant (anti-stick) properties. Any material capable of providing the desired functionality (namely, reduction of tissue sticking while simultaneously maintaining sufficient electrical transmission to permit tissue sealing) may be used as the non-stick coating, provided it has adequate biocompatibility. In some examples, the material may be porous to allow for electrical transmission.

Exemplary liquidphobic structures for use in the practice of the present invention include various chemical coatings and films. The liquidphobic structure is applied to form a coating or layer on the portions of the electrosurgical device to prevent sticking of tissue during use.

In one example, compounds that can be used to coat the electrosurgical device of the present invention can include, but are not limited to, liquidphobic compounds (including, e.g., hydrophobic, lipophobic, amphiphobic compounds, etc.). In one example, the liquidphobic structure can include fluorosilane containing compounds. For example, the fluorosilane containing compounds can include, but is not limited to, PEPE.

The application of the non-stick coating formed from the licquidphobic structure can be accomplished using any system and process capable of controlling the thickness of the coating. In some examples, the non-stick coating can be deposited by techniques including, but are not limited to, plasma deposition, painting, spraying, layering, dipping, spin-coating, applying, evaporative deposition, etc.

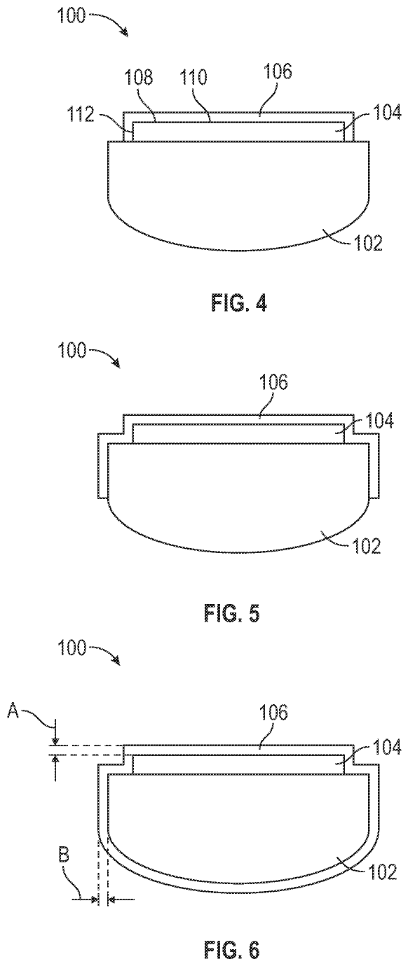

As discussed above, the thickness and the location of the non-stick coating can vary. In some embodiments, the thickness of the non-stick coating can vary such that the non-stick coating has a substantially non-uniform thickness. The thickness of the non-stick coating can be in a range of from about 10 nanometers to about 300 nanometers. A thickness of about 10 nm can provide a minimum level of non-stick performance and durability, and depending on the device and the number of intended uses, 20 nm may be more preferred. A thickness of about 300 nanometers can provide improved non-stick performance and durability over thinner coatings of the about 10-20 nm range. Above about 300 nanometers, additional performance and durability enhancements may not be realized, while additional cost is incurred. Further, depending on the particular device characteristics, coating thicknesses above 300 nm may undesirably affect electrical transmission from the tissue sealing plate to the tissue. Thus, in a possibly preferred example, the liquidphobic structure can include fluorosilane containing compounds less than the maximum of about 300 nanometers, such as having a thickness in a range from about 10 nanometers to about 200 nanometers, or more preferably in an range from about 20 nanometers to about 200 nanometers to provide the performance, durability and value. FIGS. 4 through 9 provide simplified examples of a jaw member 100 including a body portion 102 and an electrically conductive sealing plate 104 (hereinafter “sealing plate 104”) having a non-stick coating 106. The examples shown can be applied to jaw members 40, 44 discussed herein. Further, any combination of FIGS. 5 through 10 can be used to apply the non-stick coating to jaw members.

FIG. 4 illustrates an example where the non-stick coating 106 is applied to cover the entire external surface 108 of the sealing plate 104 including the tissue engaging surface 110 and the side surface 112 of the sealing plate 104. In one example, the coating thickness can be uniform or not uniform.

FIG. 5 illustrates an example where the non-stick coating 106 is applied to cover the entire external surface 108 of the sealing plate 104 including the tissue engaging surface 110 and the side surface 112 of the sealing plate 104, However, compared to the example in FIG. 4, the non-stick coating 106 extends past the sealing plate 104 and onto the body portion 102 of the jaw member 100. In one example, the coating thickness can be uniform or not uniform along the tissue engaging surface 110 and the body portion 102.

FIG. 6 illustrates an example where the non-stick coating 106 is applied to cover the entire external surface 108 of the sealing plate 104 including the tissue engaging surface 110 and the side surface 112 of the sealing plate 104. However, the non-stick coating 106 extends past the sealing plate 104 and onto the entire body portion 102 of the jaw member 100, As illustrated in FIG. 6, a first portion of the non-stick coating can have a thickness A. The non-stick coating along a majority of the tissue engaging surface 110 of the electrically conductive sealing plates can have the non-stick coating thickness A, and portions of the non-stick coating not on the tissue engaging surface of the electrically conductive sealing plates can have thickness B, which is different from thickness A. In one example, thickness A can be less than thickness B. In another example, thickness A can be greater than thickness B. Thus, the example shown in FIG. 6 illustrates the sealing plate 140 having a different thickness compared to the thickness of the coating on the jaw body 102.

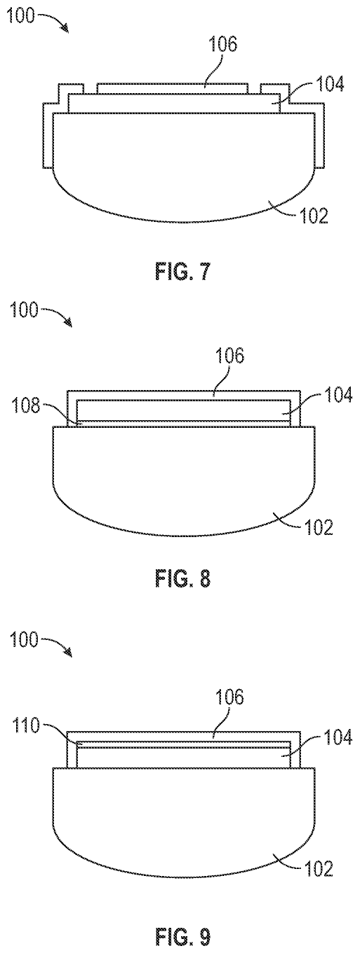

FIG. 7 illustrates an example where the non-stick coating 106 is applied to cover a portion of the external surface 108 of the sealing plate 104 and a portion of the jaw body 102. For example, the non-stick coating along the sealing plate 104 can include portions that do not include any non-stick coating, i.e., discontinuous.

FIG. 8 illustrates an example similar to the example shown in FIG. 5 but an insulative layer 108 is disposed between the sealing plate 104 and the jaw body 102. The insulative layer can be formed from polyimide. However, in other examples, any suitable electrically insulative material may be utilized, such as polycarbonate, polyethylene, etc.

FIG. 9 illustrates an example similar to the example shown in FIG. 5 but a first coating 110 is deposited between the sealing plate 104 and the non-stick coating 106. The first coating 100 can be a material that can increase the hardness and/or durability of the device. In one example, the first coating 110 can be formed from one of chromium nitride and titanium nitride. In one example, the thickness of the TiN or the CrN coating can be around 150 microns. While, e.g., the TiN is a slight electrical insulator compared to the steel of the sealing plate, it is negligible and would have little effect on the overall resistance with the non-stick coating 106 formed from a siloxane coating.

The benefits of the systems and methods of the present disclosure can include tissue adherence resistance with non-stick coatings formed from liquidphobic structures.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more,” In this document, the term “or” is used to refer to a nonexclusive or, such that “A or B” includes “A but not B,” “B but not A,” and “A and 13,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt iii to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### Exemplary Aspects.

The following exemplary aspects are provided, the numbering of which is not to be construed as designating levels of importance:

Aspect 1 provides an electrosurgical device, comprising:

at least one jaw member having an electrically conductive tissue sealing plate configured to operably couple to a source of electrosurgical energy for treating tissue; and

a non-stick coating formed from a liquidphobic structure disposed on at least a portion of the electrically conductive tissue sealing plate.

Aspect 2 provides the electrosurgical device of Aspect 1, wherein the liquidphobic structure includes a coating including a fluorosilane containing compound.

Aspect 3 provides the electrosurgical device of Aspect 2, wherein the flourosilane containing compound is perfluoropolyether (PFPE).

Aspect 4 provides the electrosurgical device of Aspect 3, wherein the non-stick coating has a thickness of 20 nanometers to 200 nanometers.

Aspect 5 provides the electrosurgical device according to any one of Aspects 1 through 4, wherein the non-stick coating has a substantially uniform thickness.

Aspect 6 provides the electrosurgical device according to any one of Aspects 1 through 4, wherein the non-stick coating has a non-uniform thickness.

Aspect 7 provides the electrosurgical device according to any one of Aspects 1 through 6, wherein the non-stick coating is discontinuous.

Aspect 8 provides the electrosurgical device according to any one of Aspects 1 through 6, wherein the non-stick coating is continuous.

Aspect 9 provides the electrosurgical device according to any one of Aspects 1 through 8, further comprising an insulative layer disposed on at least a portion of the tissue sealing plate.

Aspect 10 provides the electrosurgical device according any one of Aspects 1 through 9, wherein the non-stick coating is disposed on at least a portion of the at least one jaw member.

Aspect 11 provides the electrosurgical device according to any one of Aspects 1 through 10, wherein the tissue sealing plate is formed of stainless steel.

Aspect 12 provides an electrosurgical device, comprising:

a pair of opposing jaw members, each of the opposing jaw members including:
an electrically conductive tissue sealing plate configured to operably couple to a source of electrosurgical energy for treating tissue;
a support base configured to support the tissue sealing plate; and
an insulative housing configured to secure the tissue sealing plate to the support base; and

a non-stick coating disposed on at least a portion of at least one of the opposing jaw members, the non-stick coating formed from a liquidphobic structure.

Aspect 13 provides the electrosurgical device of Aspect 12, wherein the liquidphobic structure includes a coating including a fluorosilane containing compound.

Aspect 14 provides the electrosurgical device according to any one of Aspects 12 and 13, wherein the flourosilane containing compound is perfluoropolyether (PFPE).

Aspect 15 provides the electrosurgical device of Aspect 14, wherein the non-stick coating has a thickness of about 10 nanometers to about 200 nanometers.

Aspect 16 provides the electrosurgical device according to any one of Aspects 12 through 15, wherein the non-stick coating disposed on at least a portion of each of the tissue sealing plate, the support base, and the insulative housing.

Aspect 17 provides the electrosurgical device according to any one of Aspects 12 through 16, wherein the non-stick coating has a substantially uniform thickness.

Aspect 18 provides the electrosurgical device according to any one of Aspects 12 through 16, wherein the non-stick coating has a non-uniform thickness.

Aspect 19 provides the electrosurgical device according to any one of Aspects 12 through 18, wherein the non-stick coating is discontinuous.

Aspect 20 provides the electrosurgical device according to any one of Aspects 12 through 18, wherein the non-stick coating is continuous.

Aspect 21 provides an electrosurgical device, comprising:

a pair of opposing jaw members, each of the opposing jaw members including:
an electrically conductive tissue sealing plate configured to operably couple to a source of electrosurgical energy for treating tissue;
a support base configured to support the tissue sealing plate; and
an insulative housing configured to secure the tissue sealing plate to the support base;

a first coating disposed on at least a portion of the electrically conductive tissue sealing plate of at least one of the opposing jaw members, the first coating configured to increase the durability of the at least one of the opposing jaw members; and

a non-stick coating disposed on at least a portion of at least one of the opposing jaw members including a portion of the first coating, the non-stick coating formed from a liquidphobic structure.

Aspect 22 provides the electrosurgical device of Aspect 21, wherein the liquidphobic structure includes a coating including a fluorosilane containing compound.

Aspect 23 provides the electrosurgical device according to any one of Aspects 21 and 22, Wherein the first coating is selected from chromium nitride and titanium nitride.

Aspect 24 provides a method of manufacturing an electrosurgical device; the method comprising:

coupling an electrically conductive sealing plate to a support base to form a jaw member; and

applying a non-stick coating over at least a portion of the electrically conductive sealing plate, wherein the non-stick coating reduces sticking of the tissue to the electrically conductive sealing plate as compared to a non-coated electrically conductive sealing plate during delivery of electrosurgical energy, wherein the non-stick coating is formed from a liquidphobic structure.

Aspect 25 provides the method of Aspect 24, wherein the liquidphobic structure includes a coating including a fluorosilane containing compound.

Aspect 26 provides the method according to any one of Aspects 24 and 25, wherein the flourosilane containing compound is perfluoropolyether (PFPE).

Aspect 27 provides the method according to any one of Aspects 24 through 26, further comprising:

overmolding an insulative mated al about the support base to secure the electrically conductive sealing plate thereto.

Aspect 28 provides the method according to any one of Aspects 24 through 27, further comprising:

coupling an electrical lead to the electrically conductive sealing surface, the electrical lead configured to connect the electrically conductive sealing surface to an energy source.

Aspect 29 provides a method of manufacturing an electrosurgical device, the method comprising:

applying a first coating to at least a portion of an electrically conductive sealing plate to form a coated electrically conductive sealing plate;

coupling the coated electrically conductive sealing plate to a support base to form a jaw member; and

applying a non-stick coating over at least a portion of the coated electrically conductive sealing plate, wherein the non-stick coating reduces sticking of the tissue to the electrically conductive sealing plate as compared to a non-coated electrically conductive sealing plate during delivery of electrosurgical energy, wherein the non-stick coating is formed from a liquidphobic structure.

Aspect 30 provides the method of Aspect 29, wherein the first coating is selected from chromium nitride and titanium nitride.

Aspect 31 provides the method according to any one of Aspects 29 and 30, wherein the liquidphobic structure includes a coating including a fluorosilane containing compound.

Aspect 32 provides the method according to any one of Aspects 29 though 31, wherein the flourosilane containing compound is perfluoropolyether (PFPE).

Aspect 33 provides the method according to any one of Aspects 29 through 32, further comprising:

overmolding an insulative material about the support base to secure the electrically conductive sealing plate thereto.

Aspect 34 provides the method according to any one of Aspects 29 through 33, further comprising:

coupling an electrical lead to the electrically conductive sealing surface, the electrical lead configured to connect the electrically conductive sealing surface to an energy source.

What is claimed is:

1. An electrosurgical device, comprising:
at least one jaw member having an electrically conductive tissue sealing plate configured to operably couple to a source of electrosurgical energy for treating tissue; and
a non-stick coating formed from a liquidphobic structure disposed on at least a portion of the electrically conductive tissue sealing plate, wherein the liquidphobic structure includes a coating including a fluorosilane containing compound.

2. The electrosurgical device of claim 1, wherein the flourosilane containing compound is perfluoropolyether (PFPE).

3. The electrosurgical device of claim 2, wherein the non-stick coating has a thickness of 10 nanometers to 200 nanometers.

4. The electrosurgical device of claim 1, further comprising an insulative layer disposed on at least a portion of the tissue sealing plate.

5. The electrosurgical device of claim 1, wherein the non-stick coating is disposed on at least a portion of the at least one jaw member.

6. The electrosurgical device of claim 1, wherein the tissue sealing plate is formed of stainless steel.

7. An electrosurgical device, comprising:
a pair of opposing jaw members, each of the opposing jaw members including:
an electrically conductive tissue sealing plate configured to operably couple to a source of electrosurgical energy for treating tissue;
a support base configured to support the tissue sealing plate; and
an insulative housing configured to secure the tissue sealing plate to the support base; and
a non-stick coating disposed on at least a portion of at least one of the opposing jaw members, the non-stick coating formed from a liquidphobic structure.

8. The electrosurgical device of claim 7, wherein the liquidphobic structure includes a coating including a fluorosilane containing compound.

9. The electrosurgical device of claim 7, wherein the flourosilane containing compound is perfluoropolyether (PFPE).

10. The electrosurgical device of claim 9, wherein the non-stick coating has a thickness of about 10 nanometers to about 200 nanometers.

11. The electrosurgical device 7, wherein the non-stick coating disposed on at least a portion of each of the tissue sealing plate, the support base, and the insulative housing.

12. The electrosurgical device of claim 7, wherein the non-stick coating has a substantially uniform thickness.

13. The electrosurgical device of claim 7, wherein the non-stick coating has a non-uniform thickness.

14. The electrosurgical device of claim 7, wherein the non-stick coating is discontinuous.

15. The electrosurgical device of claim 7, wherein the non-stick coating is continuous.

16. A method of manufacturing an electrosurgical device, the method comprising:
coupling an electrically conductive sealing plate to a support base to form a jaw member; and
applying a non-stick coating over at least a portion of the electrically conductive sealing plate, wherein the non-stick coating reduces sticking of the tissue to the electrically conductive sealing plate as compared to a non-coated electrically conductive sealing plate during delivery of electrosurgical energy, wherein the non-stick coating is formed from a liquidphobic structure.

17. The method of claim 16, wherein the liquidphobic structure includes a coating including a fluorosilane containing compound.

18. The method of claim 16, wherein the flourosilane containing compound is perfluoropolyether (PFPE).

19. The method of claim 16, further comprising:
overmolding an insulative material about the support base to secure the electrically conductive sealing plate thereto.

20. The method of claim 16, further comprising:
coupling an electrical lead to the electrically conductive sealing surface, the electrical lead configured to connect the electrically conductive sealing surface to an energy source.
